(19) United States
(12) Patent Application Publication
(10) Pub. No.: US 2020/0352675 A1
(43) Pub. Date: Nov. 12, 2020

(54) SURGICAL DEVICES AND METHODS OF USE THEREOF

(71) Applicant: Body Vision Medical Ltd., Ramat Hasharon (IL)

(72) Inventor: Dorian Averbuch, Ramat Hasharon (IL)

(21) Appl. No.: 16/820,280

(22) Filed: Mar. 16, 2020

Related U.S. Application Data
(63) Continuation of application No. 15/038,849, filed on May 24, 2016, now abandoned.
(60) Provisional application No. 61/908,297, filed on Nov. 25, 2013.

(51) Int. Cl. A61B 90/00; A61B 34/10; A61B 5/06; A61B 6/00; A61B 17/00

(52) U.S. Cl. CPC A61B 90/39; A61M 25/04; A61B 5/062; A61B 5/064; A61B 6/487; A61B 17/00234; A61B 2017/00809; A61B 2090/3912; A61B 2090/3945; A61B 2034/107; A61B 2090/365; A61B 2090/3925; A61B 2090/3941; A61B 2090/3966; A61B 2017/00296; A61B 34/10

(57) ABSTRACT

In some embodiments, the instant invention provides a device including: (a) at least one sleeve composed of a material configured to exhibit a sufficient flexibility to reduce tissue damage; (b) a locking mechanism configured to secure the at least one sleeve into a position complementary to a bronchial airway; (c) a plurality of locatable elements; and where the size of the at least one sleeve is sufficient to be introduced into the bronchial airway.

### RELATED APPLICATION

This application claims the priority of U.S. provisional application Ser. No. U.S.S.N. 61/908,297, entitled “AVATS AUGMENTED VIDEO ASSISTED SURGERY,” filed Nov. 25, 2013, which is incorporated herein by reference in its entirety for all purposes.

### FIELD OF THE INVENTION

The embodiments of the present invention relate to surgical devices and methods of use thereof

### BACKGROUND OF INVENTION

Use of video-assisted thoracic surgery (VATS) during endoscopic surgery, as well as other fields of surgery, can be used during the treatment of various respiratory diseases.

### BRIEF SUMMARY OF INVENTION

In some embodiments, the instant invention provides a device including: (a) at least one sleeve composed of a material configured to exhibit a sufficient flexibility to reduce tissue damage; (b) a locking mechanism configured to secure the at least one sleeve into a position complementary to a bronchial airway; (c) a plurality of locatable elements; and where the size of the at least one sleeve is sufficient to be introduced into the bronchial airway. In some embodiments, the device further includes at least one anchoring element, where the at least one anchoring element is near or at an end of the at least one sleeve. In some embodiments, the at least one anchoring element is selected from the group consisting of: a self-extendable stent, an inflatable balloon, and an anchoring hook. In some embodiments, the plurality of locatable elements is selected from the group consisting of: radiopaque material elements, ultrasound markers, electromagnetic sensors, NIR visible markers, NIR fluorophore indocyanine green markers, LED-based markers, and any combination thereof. In some embodiments, the radiopaque material elements are selected from the group consisting of: balls, cones, spirals, and any combination thereof. In some embodiments, the material is selected from the group consisting of hydrogel, reverse thermogelling polymer, and foam-based colloid. In some embodiments, the device further includes a fixation mechanism configured to control movement of the at least one sleeve. In some embodiments, the fixation mechanism does not utilize a base to control movement of the at least one sleeve. In some embodiments, the locatable element is configured to be used with an endo-bronchial tube.

In some embodiments, the instant invention provides a method, including: (a) using data from a preoperative imaging modality to plan a procedure selected from the group consisting of a therapeutic procedure, a surgical procedure and a diagnostic procedure; (b) placing a device in a lung of a patient, including: (i) at least one sleeve composed of a material configured to exhibit a sufficient flexibility to reduce tissue damage; (ii) a locking device configured to secure the at least one sleeve into a position complementary to a bronchial airway; and (iii) a plurality of locatable elements, where the size of the at least one sleeve is sufficient to be introduced into a bronchial airway of a lung, where the lung is deflated, or at least partially inflated; (c) locking the at least one sleeve by use of the locking device in the bronchial airway of the lung; (d) operating an inter-operative imaging modality for generating medical imaging result; and (e) providing augmented video image to a practitioner. In some embodiments, the method further includes anchoring the device to reduce movement within the bronchial airway.

In some embodiments, the instant invention provides a method, (a) placing a device in a lung of a patient, including: (i) at least one sleeve composed of a material configured to exhibit a sufficient flexibility to reduce tissue damage; (ii) a locking device configured to secure the at least one sleeve into a position complementary to a bronchial airway; and (iii) a plurality of locatable elements; where the size of the at least one sleeve is sufficient to be introduced into a bronchial airway of a lung, where the lung is deflated, or at least partially inflated; (b) using data from a preoperative imaging modality to plan a procedure selected from the group consisting of a therapeutic procedure, a surgical procedure and a diagnostic procedure; (c) locking the at least one sleeve by use of the locking device in the bronchial airway of the lung; (d) operating an inter-operative imaging modality for generating medical imaging result; and (e) providing augmented video image to a practitioner.

### BRIEF DESCRIPTION OF THE DRAWINGS

The present invention will be further explained with reference to the attached figures. The figures constitute a part of this specification and include illustrative embodiments of the present invention and illustrate various objects and features thereof. Specific functional details disclosed herein are not to be interpreted as limiting, but merely as a representative basis for teaching one skilled in the art to variously employ the present invention.

FIG. 1 is a flow chart illustrating an embodiment of the present invention, showing a surgical and diagnostic procedure flow chart.

FIG. 2 is an illustration of an embodiment of the device of the present invention showing a shape-holding locatable device configured to use a mechanical locking mechanism.

FIG. 3 is an illustration of an embodiment of the device of the present invention, showing a shape holding locatable device configured to respond to a material viscosity change.

FIGS. 4A and 4B are images showing an embodiment of the results of the device of the present invention (i.e., an augmented VATS illustration).

FIGS. 5A and 5B are images showing an embodiment of the results of the device of the present invention (i.e., an augmented VATS illustration).

### DETAILED DESCRIPTION OF VARIOUS EMBODIMENTS

The present invention will be further explained with reference to the attached drawings, wherein like structures are referred to by like numerals throughout the several views. The drawings shown are not necessarily to scale, with emphasis instead generally being placed upon illustrating the principles of the present invention. Further, some features may be exaggerated to show details of particular components.

The figures constitute a part of this specification and include illustrative embodiments of the present invention and illustrate various objects and features thereof. Further, the figures are not necessarily to scale, some features may be exaggerated to show details of particular components. In addition, any measurements, specifications and the like shown in the figures are intended to be illustrative, and not restrictive. Therefore, specific structural and functional details disclosed herein are not to be interpreted as limiting, but merely as a representative basis for teaching one skilled in the art to variously employ the present invention.

Among those benefits and improvements that have been disclosed, other objects and advantages of this invention will become apparent from the following description taken in conjunction with the accompanying figures. Detailed embodiments of the present invention are disclosed herein; however, it is to be understood that the disclosed embodiments are merely illustrative of the invention that may be embodied in various forms. In addition, each of the examples given in connection with the various embodiments of the invention which are intended to be illustrative, and not restrictive. Any alterations and further modifications of the inventive feature illustrated herein, and any additional applications of the principles of the invention as illustrated herein, which would normally occur to one skilled in the relevant art and having possession of this disclosure, are to be considered within the scope of the invention.

Throughout the specification and claims, the following terms take the meanings explicitly associated herein, unless the context clearly dictates otherwise. The phrases “in one embodiment” and “in some embodiments” as used herein do not necessarily refer to the same embodiment(s), though it may. Furthermore, the phrases “in another embodiment” and “in some other embodiments” as used herein do not necessarily refer to a different embodiment, although it may. Thus, as described below, various embodiments of the invention may be readily combined, without departing from the scope or spirit of the invention.

In addition, as used herein, the term “or” is an inclusive “or” operator, and is equivalent to the term “and/or,” unless the context clearly dictates otherwise. The term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

In some embodiments, the instant invention provides a device including: (a) at least one sleeve composed of a material configured to exhibit a sufficient flexibility to reduce tissue damage; (b) a locking mechanism configured to secure the at least one sleeve into a position complementary to a bronchial airway; (c) a plurality of locatable elements; and where the size of the at least one sleeve is sufficient to be introduced into the bronchial airway. In some embodiments, the device further includes at least one anchoring element, where the at least one anchoring element is near or at an end of the at least one sleeve. In some embodiments, the at least one anchoring element is selected from the group consisting of: a self-extendable stent, an inflatable balloon, and an anchoring hook. In some embodiments, the plurality of locatable elements is selected from the group consisting of: radiopaque material elements, ultrasound markers, electromagnetic sensors, NIR visible markers, NIR fluorophore indocyanine green markers, LED-based markers, and any combination thereof. In some embodiments, the radiopaque material elements are selected from the group consisting of: balls, cones, spirals, and any combination thereof. In some embodiments, the material is selected from the group consisting of hydrogel, reverse thermogelling polymer, and foam-based colloid. In some embodiments, the device further includes a fixation mechanism configured to control movement of the at least one sleeve. In some embodiments, the fixation mechanism does not utilize a base to control movement of the at least one sleeve. In some embodiments, the locatable element is configured to be used with an endo-bronchial tube.

In some embodiments, the instant invention provides a method, including: (a) using data from a preoperative imaging modality to plan a procedure selected from the group consisting of a therapeutic procedure, a surgical procedure and a diagnostic procedure; (b) placing a device in a lung of a patient, including: (i) at least one sleeve composed of a material configured to exhibit a sufficient flexibility to reduce tissue damage; (ii) a locking device configured to secure the at least one sleeve into a position complementary to a bronchial airway; and (iii) a plurality of locatable elements, where the size of the at least one sleeve is sufficient to be introduced into a bronchial airway of a lung, where the lung is deflated, or at least partially inflated; (c) locking the at least one sleeve by use of the locking device in the bronchial airway of the lung; (d) operating an inter-operative imaging modality for generating medical imaging result; and (e) providing augmented video image to a practitioner. In some embodiments, the method further includes anchoring the device to reduce movement within the bronchial airway.

In some embodiments, the instant invention provides a method, (a) placing a device in a lung of a patient, including: (i) at least one sleeve composed of a material configured to exhibit a sufficient flexibility to reduce tissue damage; (ii) a locking device configured to secure the at least one sleeve into a position complementary to a bronchial airway; and (iii) a plurality of locatable elements; where the size of the at least one sleeve is sufficient to be introduced into a bronchial airway of a lung, where the lung is deflated, or at least partially inflated; (b) using data from a preoperative imaging modality to plan a procedure selected from the group consisting of a therapeutic procedure, a surgical procedure and a diagnostic procedure; (c) locking the at least one sleeve by use of the locking device in the bronchial airway of the lung; (d) operating an inter-operative imaging modality for generating medical imaging result; and (e) providing augmented video image to a practitioner.

In some embodiments, methods(s) of the present invention allow(s) for training inexperienced practitioners, where the training improves results, and where the learning curve of practitioners is significantly reduced. In some embodiments, the device is introduced into a lung, where the lung is deflated or partially inflated, and the device is configured to hold the lung region in a desired position and shape. In some embodiments, the device is configured to detect and/or be positioned using one or more (e.g., 2, 3, 4, etc.) imaging modalities typically available in an operation room, where the imaging modality can be a X-ray, ultrasound (US), infrared (IR), electromagnetic system, or any combination thereof.

In some embodiments, the method(s) of the present invention include(s) allowing a practitioner to perform a preoperative plan by use of a preoperative imaging modality such as, but not limited to, computerized tomography (CT) or magnetic resonance imaging (MRI). In some embodiments, the method includes superimposing a preoperative plan, e.g., on a laparoscopic video monitor, where the superimposed preoperative plan permits a practitioner to visualize a location of an internal and/or external landmark(s) such as, but not limited to, resection boundaries, lesions, incision points, critical structures, and any combination thereof. In some embodiments, the method includes sufficiently improving an efficacy and safety of a surgical procedure.

In some embodiments, device(s) of the present invention is/are configured to improve a surgical treatment and/or diagnostic outcome(s) of a video-assisted thoracic surgery (VATS).

In some embodiments, the device(s) of the present invention(s) i/ares a detectable holder device configured to permit superimposing preoperative planning elements. In some embodiments, the superimposed preoperative planning elements include at least one preoperative image over video acquired by endoscopic tools used for diagnostic and treatment. In some embodiments, the superimposed preoperative planning elements do not include at least one preoperative image over video acquired by endoscopic tools used for diagnostic and treatment.

In some embodiments, the method(s) of the present invention(s) includes using a detectable holder device permitting superimposing preoperative planning elements with or without at least one preoperative image over video acquired by endoscopic tools used for diagnostic and treatment.

In some embodiments, the method of the present invention(s) includes allowing a direct flow (i.e., streamlined the flow) of information relating to a VATS therapeutic procedure or a diagnostic procedure and reduces intervention procedure time (e.g., 20%, 30%, 40%, 50%, 60%, 70% shorter than a procedure used without device) and improves the learning curve of VATS procedure.

In some embodiments, the method of the present invention(s) includes providing improved control over a procedure/method to a practitioner (e.g., a surgeon) to allow for accurately identify a treatment area and any critical structures by use of information provided by the device, e.g., pre-operative planning images and/or high resolution imaging data.

In some embodiments, the present invention(s) is/are a device (e.g., a locatable device) configured to hold and/or support a deflated or a partially inflated lung region in a shape. In some embodiments, the device can be delivered and installed inside a lung and is configured to provide clearer access to an operation area during a surgery, where the device is configured to superimpose planning (e.g., preoperative) and/or imaging data over a real-time video, an ultrasound, fluoroscopic imaging, or any other image acquired during a minimally invasive surgery. In some embodiments, the device of the present invention can be used with one or multiple kinds of imaging modalities. In some embodiments, imaging modalities can include, but are not limited to radiography, magnetic resonance imaging (MRI), nuclear medicine, ultrasound, elastography, tactile imaging, photoacoustic imaging, thermography, tomography, echocardiography, and functional near-infrared spectroscopy.

In some embodiments, the method(s) of the present invention(s) include(s) improving a real-time image acquired during surgery by placing additional information on the real-time image, where the additional information is sourced from at least one pre-operative image and/or planning data. In some embodiments, the method(s) allow for integrating the patient-related/derived data and imaging data for the purpose of improving an outcome of a diagnostic or surgical procedure. In some embodiments, the method(s) of the present invention include(s) assisting the diagnostic and therapeutic procedures in lung, liver and kidney.

In some embodiments, the device(s) of the present invention is/are configured to allow a practitioner to navigate and/or operate a medical instrument based on the information provided by a superimposed anatomical and/or planning data image, where the anatomical and/or planning data image was extracted from a high quality pre-operational image and/or a combination of pre-operational image with additional imaging sources available in the operation room.

In some embodiments, the device(s) of the present invention is/are configured to provide an augmented real-time image of an actual surgical instrument and operated tissue together with a targeted anatomical feature(s) and planning information, for example, but not limited to, incision points, cutting area boundaries, reference points, or any combination thereof.

In some embodiments, the method(s) of the present invention include(s) combining multimodal imaging information and/or utilizing previously acquired high quality volume data of inflated lung during the surgery performed on the collapsed or partially inflated lung.

In some embodiments, the method(s) of the present invention include(s) utilizing the decisions performed by practitioner on pre-operative data in an efficient way, when the most visually informative combination of the elements displayed on the screen, their resolution and quality can be dynamically determined (e.g., presented in real-time to a practitioner) on an application-specific or user-specific basis.

In some embodiments, the device(s) of the present invention include(s) a shape-holding locatable device configured to preserve the shape of a lung and/or lobe area (e.g., a portion of a lung or lobe); and provide detectability through the known imaging modalities, where the imaging modalities are typically available in the operation room. In some embodiments, imaging modalities can include at least one of the following modalities: C-Arm, Ultrasound, Infrared, electromagnetic localization system, or any combination thereof. In some embodiments, the device of the present invention includes a combination of (a) endo-bronchial tube and (b) a shape-holding device.

In some embodiments, the method(s) of the present invention(s) include(s) using an apparatus for maintaining an accurate registration between operative real-time video images, single or multiple catheter localization modality/(ies), static preoperative images, or any combination thereof.

In some embodiments, the device(s) of the present invention(s) can be an endoscopic device(s) configured to integrate/combine (1) a video image (e.g., a conventional video image obtained by, e.g., digital) with (2) a localization modality configured to provide an image to a practitioner. In some embodiments, the device further includes a locatable catheter configured to provide an image in real time over video image that can include information comprised from preoperative images.

In some embodiments, the method(s) of the present invention include(s) navigating a surgical instrument to an area of interest in a lung, where the area is percutaneous and endobronchial.

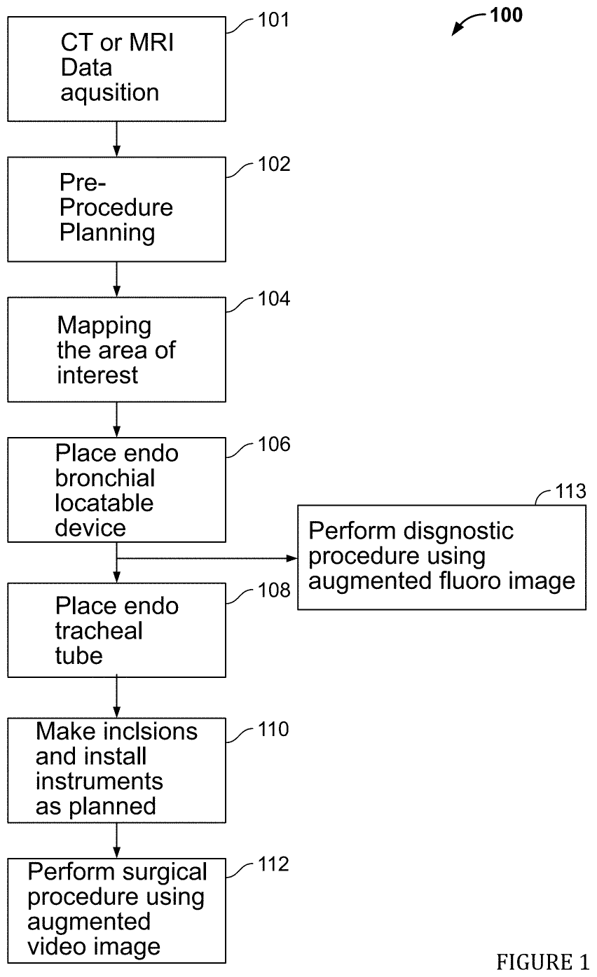

FIG. 1 illustrates a flowchart showing an embodiment of a method of the present invention 100. At 101 of the method 100, CT or MRI data is acquired and transformed into a 3D volume, which can be used during a surgical treatment and/or a diagnostic procedure to plan a treatment and/or diagnosis, and the CT or MRI data can be superimposed (i.e., the desired information, which can be a portion of a CT or MRI data) over (e.g., layered on top) the real-time video image. At 102 of the method 100, the practitioner, such as, e.g., a surgeon, can perform a pre-procedure planning on the pre-procedure data acquired at 101, during which the practitioner can mark the a plurality of: (1) incision points for tool introduction, (2) areas of interest (e.g., the boundaries of the area to resect around the suspicious lesion), (3) critical structures (e.g., major blood vessels, restricted area), and (4) the setup and position for locatable catheter. In an embodiment, the method can be performed manually or semi-automatically, such as, e.g., when a portion of information is automatically identified by computer software. Once the planning is completed, at 104 the information is processed for generating visualization data or elements that can be displayed during surgical procedure. In some embodiments, a pathway to introduce a shape-holding locatable device can be generated and/or stored. In some embodiments, instructions (e.g., guidance instructions) based on anatomical knowledge and procedure details are automatically generated and presented to the practitioner.

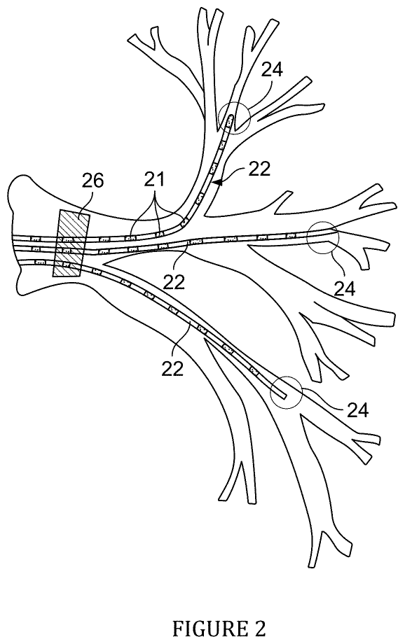

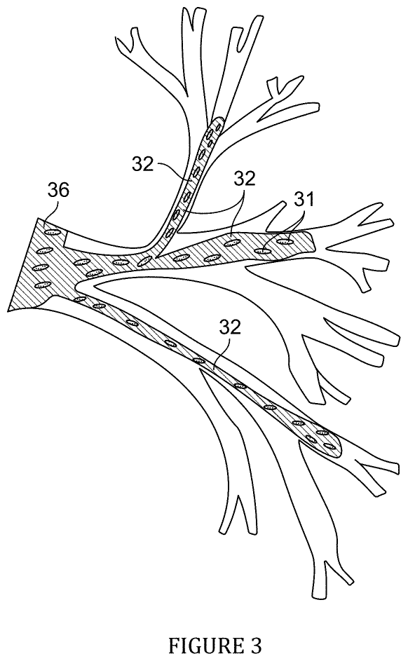

In an embodiment of the method(s) of the present invention, the bronchoscopy is performed as illustrated in 106, where the guidance instructions direct the user to place the base 26 (FIG. 2) or 36 (FIG. 3) of shape-holding locatable device at an anatomically significant bifurcation. In an embodiment, additional directions can guide a practitioner to insert/place the at least one sleeve of the shape-holding locatable device 22 (FIG. 2) or 32 (FIG. 3) at each or several bronchial airway(s), where the at least one sleeve splits out from a base location. In an embodiment, the method allows a practitioner to illuminate an at least one area inside the lung.

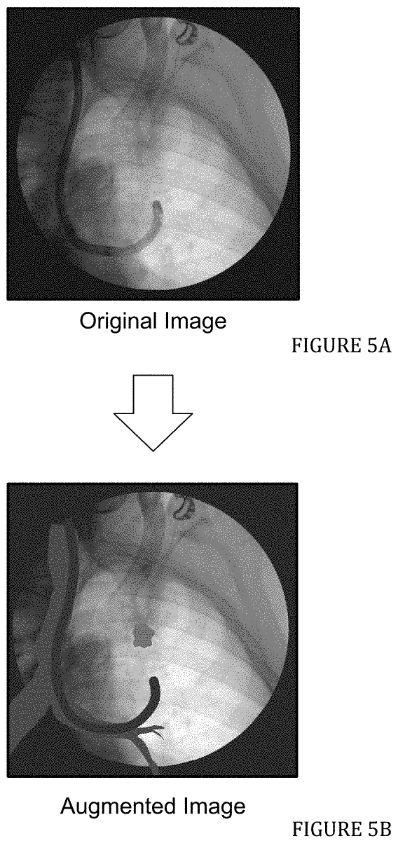

In an embodiment of the method(s) of the present invention(s), the diagnostic procedure 113 is performed using an augmented video image. As used herein, an “augmented video image” refers to an image that superimposes planning and/or anatomical information over a real-time conventional fluoroscopic image(s) of, e.g., a diagnostic bronchoscopy. In an embodiment, the localization modality can be operated periodically and/or continuously during a diagnostic procedure. FIGS. 5A and 5B show examples of an embodiment, showing a result of an augmented fluoroscopic image. This example (FIG. 5A, 5B) of an embodiment of the method of the present invention illustrates the comparison between a fluoroscopic image (as shown in FIG. 5A) and augmented fluoroscopic image (as shown in FIG. 5B) where the anatomical elements, such as, e.g., the bronchial airway, are highlighted together with the suspicious lesion area.

In an embodiment of the method(s) of the present invention, the shape-holding locatable device can be positioned to permit the double lumen endotracheal tube to be placed according to existing practice in 108. In an embodiment, the double-lumen endotracheal tube placement is performed to achieve lung separation. The lungs are paired organs interconnected by bronchi and trachea that function as one unit. In some embodiments, thoracic surgeons may require lung separation and one-lung ventilation to perform certain procedures and provide optimal surgical exposure. In some embodiments, procedures that require one-lung ventilation include lobectomy, pneumonectomy, pleural decortication, bullectomy, bronchopulmonary lavage, esophagogastrectomy, thymectomy, and mediastinal mass resections. In some embodiments, after completion/verification of endotracheal tube placement, the ventilation is switched to a contralateral lung for optimizing deflation of the lung that will be operated upon.

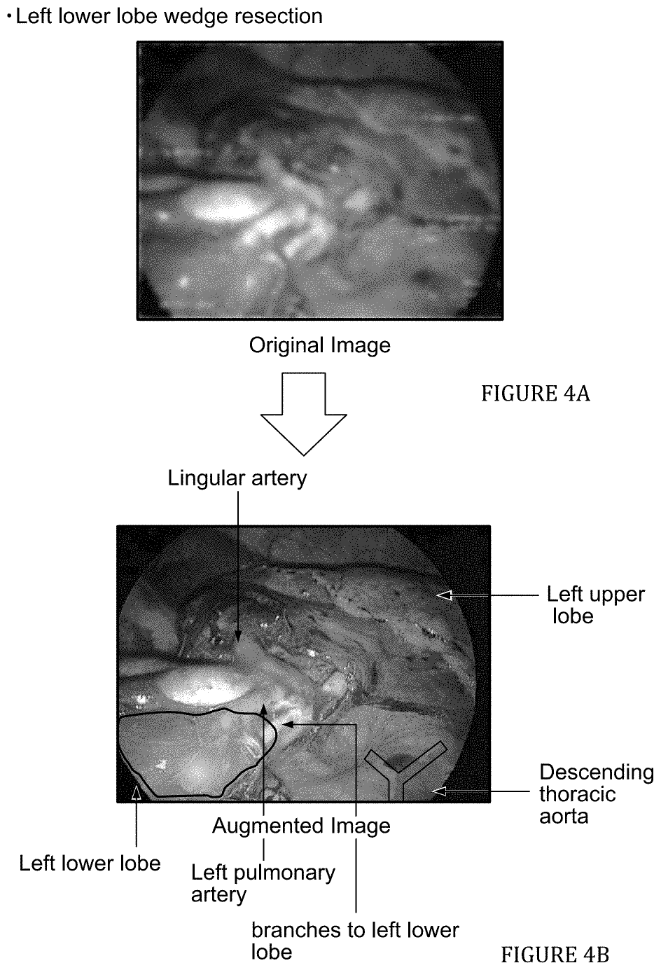

In some embodiments of the method(s) of the present invention, the incision can be performed in 110 according to preoperative planning for optimizing access to the area of interest. In some embodiments, the surgical procedure 112 can be performed using an augmented video image configured to superimpose planning and anatomical information over a real-time conventional video image(s) of a surgery, e.g., thoracic surgery. In some embodiments, the localization modality can be operated periodically or continuously during a surgical procedure. In an embodiment of the method(s) of the present invention, an illustration of a resulting augmented video image is provided by FIG. 4B, which further shows a comparison between the typical thoroscopic image (FIG. 4A) and the augmented thoroscopic image (FIG. 4B) where the anatomical elements (such as, e.g., vessels) are highlighted, as well as the lesion and resection area.

FIG. 2 and FIG. 3 are embodiments of the device of the present invention, showing non-limiting examples of design options for the shape-holding locatable device. In some embodiments, the shape holding capability is achieved through the sleeves 22 on FIG. 2 or item 32 on FIG. 3, being introduced into the bronchial airways. In some embodiments, the sleeves of the device can have at least two modes of operation: (1) flexible navigation mode that allows easy introduction (compared to, e.g., typical applications not using the device) of the catheters to desired location (e.g., region of operation/procedure), and (2) freeze mode that can allow locking the sleeves in specific shape and relative position to each other; where the certain amount of flexibility can be added to the freeze mode to reduce the tissue damage through the restriction of allowed applied forces from tissue on the catheter. In some embodiments, the reduced tissue damage measures between 10%-90% compared to the tissue damage generated by a typical procedure. In some embodiments, the reduced tissue damage measures between 30%-90% compared to the tissue damage generated by a typical procedure. In some embodiments, the reduced tissue damage measures between 60%-90% compared to the tissue damage generated by a typical procedure. In some embodiments, the reduced tissue damage measures between 10%-60% compared to the tissue damage generated by a typical procedure. In some embodiments, the reduced tissue damage measures between 10%-30% compared to the tissue damage generated by a typical procedure.

In an embodiment, FIG. 2 shows a mechanical locking mechanism of the device of the present invention. In an embodiment, FIG. 3 shows a material configured to change at least one property, such as, e.g., viscosity from liquid to the solid state under controlled physical environment configuration (and vise versa), and is further configured to allow efficient installation of the device before a procedure and device removal after a procedure. In some embodiments, examples of materials configured to change at least one property can be selected from a group including: a hydrogel, a reverse thermo-gelling polymer, and a foam-based colloid.

In some embodiments of the device(s) of the present invention(s), the sleeve(s) of a shape-holding locatable device can include a working channel. In some embodiments, the working channel can include an elastic outer bag. In an embodiment, the sleeve(s) can be configured to provide specific tags detectable through localization modality.

In some embodiments, the device of the present invention includes an anchoring mechanism (24 on FIG. 2), where the anchoring mechanism is configured to maintain the device in a specific location (e.g., area of where a surgical procedure will take place). In some embodiments, anchoring mechanisms are selected from a group consisting of, but are not limited to: a self-extendable stent, an inflatable balloon, and an anchoring hook.

In an embodiment, FIG. 2 (21) or FIG. 3 (31) illustrates the locatable element feature of the shape-holding locatable device of the present invention. In some embodiments, the locatable element feature is configured to depend on a localization modality. In an embodiment, the locatable element can be a single element or a combination of elements—for multi-modal localization, including the following: (1) radiopaque material balls, cones, spirals etc., (2) ultrasonic markers, (3) electromagnetic sensors of 5 degrees of freedom (DOFs) or 6 DOFs, (4) NIR visible markers or NIR fluorophore indocyanine green (IGC), (5) LED-based markers, or any combination thereof.

In some embodiments, the method(s) of the present invention allow(s) reusing efficiently pre-procedure imaging modality and planning information through generation of an augmented video image, where the augmented video image can include additional imaging and/or planning information superimposed over it comprising: (a) using first imaging modality to obtain at least one image of lung in specific known or predefined breathing condition; (b) planning (manual or automatic) a procedure through defining landmarks, such as, but not limited to: area of interest, incision points, critical structures, bifurcations, anatomical organs, (c) installing a locatable shape-holding device and/or the elements of the locatable shape-holding device inside a natural body cavity such as, e.g., a bronchial airway; (d) registering at least one image, a portion of the at least one image or an image-based planning information sourced from a first imaging modality and at least one image from a localization second imaging modality, where a multimodal localization of the second imaging modality is comprised from two or more localization modality sources; (e) registering at least one image of the second imaging localization modality to a third imaging modality being used for diagnostic bronchoscopy and/or VATS; (f) superimposing at least one image, a portion of the at least one image or image based planning information sourced from first imaging modality with any combination of second localization imaging modality and third vats imaging modality; navigating a diagnostic and/or surgical instrument(s) in percutaneous and/or endobronchial ways to perform surgery utilizing augmented planning information superimposed over real time and/or static images acquired during surgical procedure.

In some embodiments, the device(s) of the present invention include a locatable shape-holding device for navigation and installation inside the natural body cavities comprising: (a) a base to be installed at the edge of the area of desirable shape preservation; (b) at least one sleeve, where the at least one sleeve comprises at least two states of operation: (i) flexible and (ii) stiff with predesigned stiffness level and movement tolerance relatively to the base; (c) at least one locatable element; (d) an outer elastic bag; (e) a working channel; or any combination thereof.

In some embodiments, the device(s) of the present invention include a locatable shape-holding device for navigation and installation inside the natural body cavities comprising: (a) a base to be installed at the edge of the area of desirable shape preservation; (b) at least one sleeve, where the at least one sleeve comprises at least two states of operation: (i) flexible and (ii) stiff with predesigned stiffness level and movement tolerance relatively to the base; (c) at least one locatable element; (d) an outer elastic bag; or any combination thereof.

In some embodiments, the device(s) of the present invention include a locatable shape-holding device for navigation and installation inside the natural body cavities comprising: (a) a base to be installed at the edge of the area of desirable shape preservation; (b) at least one sleeve, where the at least one sleeve comprises at least two states of operation: (i) flexible and (ii) stiff with predesigned stiffness level and movement tolerance relatively to the base; (c) at least one locatable element; or any combination thereof.

In some embodiments, the device(s) of the present invention are configured to maintain a shape (e.g., a desired shape for optimizing results, i.e., a clearer image) of organ or organ portion. In some embodiments, the shape of a deflated area of lung can be inflated close or similar to the lung inflated by use of/during, e.g., VATS. In some embodiments, an anchoring mechanism is configured to allow each sleeve to be fixed in place relative to the organ tissue at, e.g., but not limited to, a tip of a designated organ tissue or other given location. In some embodiments, the device does not include a base. In some embodiments, the device includes a fixation mechanism configured to allow setting and maintaining mutual relative movement of the sleeve(s). In some embodiments, the states of operation of the device are achieved through mechanical element assembly or material state changes. In some embodiments, each sleeve is configured to display a unique identification pattern that can be optionally used during localization and registration. In some embodiments, the at least one sleeve is configured to provide at least one working channel for introduction of diagnostic, navigation, and/or treatment tools into an organ and/or organ tissue. In some embodiments, the operation state can be selected for each sleeve independent of any other sleeve. In some embodiments, the operation state is set for the device as a whole. In some embodiments, the device includes a locatable shape-holding device and an endo-bronchial tube.

In some embodiments of the method(s) of the present invention, the anatomical information is obtained by a preoperative imaging modality selected from the group consisting of CT scan or chest X-ray scan and the real-time instrument information inter-procedure imaging modality is obtained by (or at least one of the following) fluoroscopic imaging, CT, cone-beam CT, bronchoscopic imaging, laparoscopic imaging or ultrasound, or any combination thereof. In some embodiments, the method can be a diagnostic, a pre-therapeutic or a therapeutic medical procedure occurring in a lung. In some embodiments, the preoperative imaging can be obtained before the procedure or during a procedure. In some embodiments, the lung can be deflated, partially inflated (or at least partially inflated), fully inflated, or breathing.

In some embodiments of the method(s) of the present invention, the registration is performed between the device image(s) detected from inter-procedure imaging modality and correspondent portion of the bronchial airway from preoperative imaging modality. In some embodiments, the registration further includes correspondent anatomical features such as, but not limited to, bifurcations, pathways, airway centerlines. In some embodiments, the placing of the device in lung is performed prior to creating data by primary imaging modality.

In some embodiments, the registration is performed between any combinations of correspondent pairs such as device image(s), anatomical features, from preoperative imaging modality and inter-procedure imaging modality. In some embodiments, the registration between preoperative imaging modality and inter-procedure imaging modality is achieved by use of a device. In some embodiments, the augmented video image comprises anatomical information from a preoperative imaging modality and real-time instrument information from inter-procedure imaging modality.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

1-14. (canceled)

15. A device, comprising:
a) at least two sleeves sized to enable the at least two sleeves to be introduced into a bronchial airway of a lung of a patient;
b) a locking mechanism configured to allow setting and maintaining mutual relative movement of the at least two sleeves, wherein the locking mechanism is capable of selectively and reversibly placing the at least two sleeves in either (1) a navigation mode in which the at least two sleeves are flexible, thereby to allow the at least two sleeves to be introduced into the bronchial airway, or (2) a freeze mode in which the at least two sleeves hold a specific shape and retain a relative position to one of the at least two sleeves with respect to another of the at least two sleeves, to allow the at least two sleeves to be relatively retained in place within the bronchial airway;
c) a fixation mechanism configured to control relative movement of the at least two sleeves; and
d) a plurality of locatable elements.

16. The device of claim 15, further comprising at least one anchoring element, where the at least one anchoring element is near or at an end of the at least two sleeves.

17. The device of claim 16, wherein the at least one anchoring element is selected from the group consisting of: a self-extendable stent, an inflatable balloon, and an anchoring hook.

18. The device of claim 15, wherein the plurality of locatable elements is selected from the group consisting of: radiopaque material elements, ultrasound markers, electromagnetic sensors, NIR visible markers, NIR fluorophore indocyanine green markers, LED-based markers, and any combination thereof.

19. The device of claim 18, wherein the radiopaque material elements are selected from the group consisting of: balls, cones, spirals, and any combination thereof.

20. The device of claim 15,
wherein the sleeve includes a material that is configured to change at least one property based on a physical environment, and
wherein the material is selected from the group consisting of hydrogel, reverse thermogelling polymer, and foam-based colloid, and
wherein the locking mechanism is configured to change the physical environment thereby to change the at least one property of the material.

21. The device of claim 20, wherein the fixation mechanism does not utilize a base to control movement of the at least two sleeves.

22. The device of claim 15, wherein the locatable element is configured to be used with an endo-bronchial tube.

23. The device of claim 15, wherein the at least two sleeves in the freeze mode are configured to retain the lung in a desired position when the lung is in a deflated state, whereby a clinician can perform a surgical procedure within the lung.
